(19) United States
(12) Patent Application Publication
(10) Pub. No.: US 2004/0253210 A1
(43) Pub. Date: Dec. 16, 2004

(54) Adenovirus type7 vectors

(76) Inventors: Marjorie Robert-Guroff, Rockville, MD (US); Xinli Nan, Bethesda, MD (US); Bo Peng, Rockville, MD (US); Tae-Wook Hahn, Kyeonggi-Do (KR)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/487,974

(22) PCT Filed: Aug. 29, 2002
(86) PCT No.: PCT/US02/27592

(30) Foreign Application Priority Data
Aug. 30, 2001 (US) 60316361

(51) Int. Cl. A61K 48/00; C07H 21/04; C12N 7/00; C12N 15/861

(52) U.S. Cl. 424/93.2; 435/235.1; 435/456

(57) ABSTRACT

The current invention provides novel adenovirus type 7 cosmid vectors for the production of adenovirus type 7 for use in gene transfer. In particular, the invention provides a replication-defective adenovirus type 7 that expresses one or more HIV polypeptides for use in stimulating an immune response to HIV-1.

### CROSS-REFERENCES TO RELATED APPLICATIONS

The current application claims the benefit of U.S. provisional application No. 60/316,361, filed Aug. 30, 2001, which is herein incorporated by reference

### BACKGROUND OF THE INVENTION

The present invention provides new cosmid adenovirus serotype 7 (Ad7) vectors and methods of making and using the vectors to produce Ad7 adenovirus for vaccines and gene transfer. In particular, the present invention provides new Ad7 vectors for the treatment or prevention of diseases such as HIV-1 infection.

Adenoviruses have become important tools in vaccine development and gene therapy as vectors for in vivo and ex vivo transfer of heterologous, therapeutic and/or immunogenic genes to prevent or treat diseases such as HIV infection, cancer and various genetic diseases. Adenoviruses offer several advantages, for example, they can be produced at high titers and can infect resting and nondividing cells. Furthermore, the adenoviral genome can be manipulated to accommodate foreign genes of up to about 8 kb in length. In addition, as an adenoviral vector does not insert its DNA into the chromosome of a cell, its effect is impermanent and unlikely to interfere with the cell's normal function. Lastly, live adenovirus have been safely used as a human vaccines (Horwitz, “Adenoviruses,” in Virology (Fields et al., eds, Lippincott-Raven Publishers, Philadelphia, 3rd ed., pp. 2149-2171, 1996; Berkner et al., J. Virol., 61, 1213-1220 (1987); Couch et al., Amer. Rev. Respir. Dis. 88:394-403, 1963: Franklin et al., J. Infect. Dis. 124:148-154, 1971; and Franklin et al., J. Infect. Dis. 124:155-160, 1971).

Most adenoviruses employed as vaccines or for gene therapy are group C adenoviruses, in particular Ad5 and Ad2. There are, however, limitations to the use of group C adenoviral vectors only. For example, a host can develop an immune response to the particular adenoviral vector as a result of natural exposure of the host to the same type of adenovirus prior to the initiation of treatment with an adenovirus vaccine or therapy vector. The host can also develop an immune response as a result of the exposure of the host to the adenoviral vector in the course of therapy itself. A cellular immune response can reduce the life span of cells infected with the adenoviral vector and thereby reduce the expression of the foreign nucleic acid, thereby diminishing the overall effectiveness of a vaccine. Indeed, it has been noted empirically that a major limitation of the currently used group C adenoviral gene therapy systems is the short duration of gene expression. See, e.g., Crystal et al., Nature Genetics, 8, 42-51 (1994). Moreover, a humoral immune response, resulting in the production of antibodies, can significantly reduce the effectiveness of a vaccine or therapy using a particular adenoviral vector, i.e., if the same adenovirus serotype is re-administered during a vaccination or therapeutic regimen, the host may generate neutralizing antibodies against the fiber or hexon proteins of the viral vector thus generating a serotype-specific anti-adenovirus response. There is therefore a need to develop non-group C adenoviral vectors.

Non-group C adenoviruses include adenovirus type 7 (Ad7), which is categorized as a group B adenovirus. Live adenovirus type 7 has been administered orally as enteric-coated table to control acute adenovirus respiratory disease (e.g., Franklin et al., J. Infect. Dis. 124:148-154, 1971) and was shown to be effective and safe. Vectors based on Ad7 are therefore attractive candidates for use in vaccines and gene therapy. Such vectors have been described (see, e.g., U.S. Pat. No. 5,837,551; WO01/53504 and Chanda et al., Virology 175:535-547, 1990).

To date, recombinant adenovirus type 7 vectors have typically been generated by homologous recombination. In this method, a gene of interest is cloned into a specific region of an adenovirus shuttle vector plasmid. The plasmid is co-transfected with at least one additional fragment of adenoviral DNA into a host cell. Infectious recombinant adenoviral particles are then produced through homologous recombination. This procedure, however, can be cumbersome and inefficient due to the large size of the adenoviral genome as well as lead to the generation of contaminating viral recombinants inadvertently generated during recombination. More recently, alternative methods have been developed to more efficiently produce adenovirus. These include using a cosmid vector system for the construction of recombinant adenoviral vectors so that the large adenoviral genome can be more readily manipulated (see, e.g., Fu et al., Human Gene Therapy 8:1321-1330, 1997; Kojima et al., Biochem. Biophys. Res. Comm. 246:868-872, 1998; Danthinne, et al., J. Virol. Methods 81:11-20, 1999; and Danthinne et al.,Gene Therapy 7:80-87, 2000). However, this system has only been applied to Ad5. There is a need to develop efficient vector systems for non-group C adenoviruses as well. The current invention addresses this need and provides a cosmid system to generate Ad7 cosmid vectors.

The present invention also provides Ad7 vectors for the prevention or treatment of HIV infection. An Ad7 HIV vaccine, in which the infectious Ad7 particles was generated by homologous recombination, has been previously described (see, e.g., Chanda et al., supra). The virus was replication-competent, however. For safety considerations, it is often desirable to use an adenoviral vector that is replication incompetent. The current invention therefore also provides a replication incompetent adenovirus generated using an Ad7 cosmid vector system. This Ad7 HIV vector is useful as an HIV therapeutic agent and/or vaccine.

### BRIEF SUMMARY OF THE INVENTION

The present invention provides new adenovirus serotype 7 cosmid vectors that comprise an adenovirus serotype 7 genome. The adenovirus serotype 7 genome can lack a functional E1 gene region. Often, all or part of the E1 gene region is deleted. The vector can further lack a functional region selected from the group consisting of an E2, E3, and E4 region. In some embodiments, all or part of an E3 gene region is deleted.

In a preferred embodiment, the vectors of the invention further comprises an expression cassette comprising a nucleic acid sequence encoding a heterologous gene product. In some embodiments, the expression cassette comprises a nucleic acid sequence encoding an HIV gene product, for example, an HIV envelope polypeptide. The expression cassette can also comprise other nucleic acid sequences, such as a nucleic acid sequence encoding a rev gene product.

Thus a particular adenovirus serotype 7 cosmid vector of the invention comprises an adenovirus serotype 7 genome which is deleted in all or part of an E1 region and an E3 gene region and which further comprises an expression cassette comprising a nucleic acid sequence encoding an HIV gene product such as an HIV envelope gene product. The vector can also comprise other nucleic acid sequences, for example, a nucleic acid sequence encoding an HIV rev gene product.

In another aspect, the invention provides a method of producing a replication defective adenovirus type 7, the method comprising transfecting an adenovirus type 7 packaging cell line with a cosmid vector comprising an adenovirus serotype 7 genome lacking a functional E1 gene. Typically, all or part of the E1 gene is deleted. In some embodiments, all or part of an E3 gene is also deleted. In preferred embodiments, adenoviral genome further comprises an expression cassette comprising a nucleic acid sequence encoding a heterologous gene product such as an HIV gene product, e.g., an evelope polypeptide. The expression cassette can also comprise a nucleic acid sequence comprising an additional gene product such as a cytokine or an HIV rev gene product.

In another aspect, the invention provides a method of preventing or treating infection with HIV-1, the method comprising administering a replication defective adenovirus comprising an adenovirus type 7 genome lacking a functional E1 gene region, wherein the adenovirus type 7 genome further comprises an expression cassette encoding an HIV-1 gene product. Typically, all or part of the E1 gene region is deleted. Often the adenovirus administered to prevent or treat HIV-1 infection further lacks a functional region selected from the group consisting of an E2, E3, and E4 region. Often, the viral genome is deleted in all or part of E3. Frequently, an adenovirus comprising an expression cassette encoding an HIV-1 env polypeptide is administered. In some embodiments, the expression cassette further comprises another gene product, such as an HIV rev gene product.

In other embodiments, the methods of administering adenovirus to prevent or treat HIV-1 infection further comprises administering a replication competent adenovirus. The methods can also be performed by administering an adenovirus of a different serotype.

In another aspect, the invention provides a replication defective adenovirus, wherein the adenovirus comprises an adenovirus type 7 genome which is deleted in all or part of an E1 gene region and further, wherein the adenovirus type 7 genome comprises an expression cassette comprising a nucleic acid sequence encoding an HIV gene product such as an envelope polypeptide. The adenovirus can also comprise an expression cassette encoding an additional heterologous gene product such as an HIV rev sequence.

### BRIEF DESCRIPTION OF THE DRAWINGS

FIG. 1 provides a schematic of the cloning strategy used to generate pAd7L.

FIG. 2 provides a schematic of the cloning strategy used to generate pAd7I.

FIG. 3 provides a schematic of the cloning strategy used to generate pAd7-E1 a.p.

FIG. 4 provides a schematic of the cloning strategy used to generate pAd7ΔE1aE1b.p.

FIG. 5 provides a schematic of the cloning strategy used to generate pAd7.M.

FIG. 6 provides a schematic showing the preparation of cosmid vector DNA.

FIG. 7 provides a schematic of the cloning strategy used to generate cAd7.

FIG. 8 provides a schematic of the cloning strategy used to generate cAd7ΔE3HIV_{MN}Env/Rev.

FIGS. 9A-9E provide a schematic of the cloning strategy used to generate cAd7ΔE1E3HIV_{MN}Env/Rev.

FIGS. 10A and 10B provide maps of new vectors for the construction of single or double recombinant adenoviruses.

### DETAILED DESCRIPTION OF THE INVENTION

The present invention provides novel adenovirus type 7 vectors for use as vaccine and gene therapy vectors. These vectors are cosmid-based vectors that can be produced as a single insert in a cosmid. The cosmid DNA can then be used to transfect a host cell line to produce the adenoviral particles.

Definitions

The term “adenovirus” or “Ad” includes all adenoviruses, including all members of the known six subgenera, classified as A to F. “Adenovirus type 7” or “Ad7” refers to a group B adenovirus, adenovirus serotype 7. Serotype classification is typically based on hemagglutination and resistance to neutralization by antisera to other known adenovirus serotypes. Type-specific neutralization results predominantly from antibody binding to epitopes on the virion hexon protein and the terminal knob portion of the fiber protein (see, e.g., Shenk, “Adenoviridae: The Viruses and Their Replication”, pages 2111-2148, of Fields Virology, supra).

An adenovirus can be classified as an Ad7 virus using a number of different methodologies (see, e.g., Shenk, supra and Horwitz, supra), typically, an immune assay and most often, a solid phase immunoassay. In current practice for specific adenovirus serotyping, the hemagglutination properties of a virus isolate are often determined, followed by serologic tests to inhibit hemagglutination or to neutralize the virus with type-specific antibodies (see, e.g., Horwitz, supra). Other immune assays can also be used to type an adenovirus. For example, an Ad7 adenovirus can determined using type-specific antibodies to epitopes on the hexon or fiber protein of Ad7, often the fiber protein, using an assay such as immunofluorescence to detect specific binding of the antibody to the epitope. “Specific binding” refers to a binding reaction that is determinative of the presence of Ad7 protein. Thus, under designated immunoassay conditions, the specified antibodies bind to a particular Ad7 hexon or fiber protein and do not substantially bind in a significant amount to other proteins, including other adenoviral capsid proteins. Typically a specific or selective reaction will provide a signal that is at least 10 to 100 times background binding to other proteins.

An “Ad7 genome” as used herein refers to an adenoviral 7 derived nucleic acid that encodes a type 7 adenovirus. As appreciated by one of skill in the art, an Ad7 genome of the invention need not include the complete genome, but can be deleted or inactivated in various regions as described below.

A “cosmid vector” refers to a plasmid vector that contains one or two copies of a small region of bacteriophage λ DNA, the cohesive end site (cos), which contains all of the cis-acting elements required for packaging of the DNA into bacteriophage λ particles. A cosmid vector typically contains about 45 kb of foreign DNA.

“Replication defective”, “replication incompetent”, or “replication deficient” are used interchangeably to refer to a viral genome that does not comprise all of the genetic information for the virus to replicate in cells that are not capable of complementing deleted adenoviral functions.

The term “replication competent cell” or “replication competent host cell” or “producer cell” or “packaging cell” includes any cell capable of supporting the replication of an adenoviral genome and capsid and the encapsidation process. For example, recombinant adenoviral vectors possessing a deletion of E1 gene functions are essentially unable to replicate except in cell lines that have been engineered to complement E1 functions.

The term “heterologous” when used with reference to a nucleic acid, indicates that the nucleic acid is in a vector, a cell, or a virus where it is not normally found in nature; or, comprises two or more subsequences that are not found in the same relationship to each other as normally found in nature, or is recombinantly engineered so that its level of expression, or physical relationship to other nucleic acids or other molecules in a cell, or structure, is not normally found in nature. For instance, a heterologous nucleic acid is typically recombinantly produced, having two or more sequences from unrelated genes arranged in a manner not found in nature: e.g., an HIV gene operatively linked to a promoter sequence inserted into an adenovirus-based vector of the invention.

The term “expression cassette” refers to a series of nucleic acid sequence elements that permit transcription of a gene or polynucleotide in a host cell. At a minimum, the expression cassette includes a promoter and a heterologous nucleic acid sequence. Expression cassettes can also include, e.g., transcription termination signals, polyadenylation signals, enhancer elements, and the like. Thus, falling within the definition of “expression cassette” are “expression vectors,” “cloning vectors,” “viral vectors,” and the like, all terms which usually refer to viruses, plasmids or other nucleic acid molecules that are able to transduce and/or replicate in a chosen host cell.

“Pharmaceutically acceptable” refers to a non-toxic, inert, and/or physiologically compatible composition.

A “pharmaceutical excipient” comprises a material such as an adjuvant, a carrier, pH-adjusting and buffering agents, tonicity adjusting agents, wetting agents, preservative, and the like.

Ad7 Cosmid Systems

Although cosmid systems have been used to produce recombinant AdS viruses (see, e.g., Fu et al, Human Gene Therapy 8:1321-1330, 1997; Kojima et al., Biochem. Biophys. Res. Comm. 246:868-872, 1998; Danthinne et al., J. Virol Methods 81:11-20, 1999; and Danthinne and Werth, Gene Therapy 7:80-87, 2000), an Ad7 cosmid system has not been known prior to the current invention.

The general techniques used in creating the Ad7 cosmid vectors and Ad7 viruses employ recombinant DNA methodology well known to those of skill in the art. Techniques for the manipulation of nucleic acids, such as subcloning, sequencing, hybridization, PCR, and the like are well described in the scientific and patent literature, see, e.g., Sambrook and Russell, eds, Molecular Cloning: A Laboratory Manual, 3rd Ed, vols. 1-3, Cold Spring Harbor Laboratory Press, 2001; and Current Protocols in Molecular Biology, Ausubel, ed. John Wiley & Sons, Inc. New York (1997).

All adenoviruses are morphologically and structurally similar in the general organization of the genome, although as appreciated by one of skill in the art, the actual sequence of the genome among groups is quite variable, for example, the amount of DNA sequence homology between groups is only about 10% to 15% (see, e.g., Horwitz, supra). These viruses are nonenveloped, regular icosahedrons, 70-100 nm in diameter, consisting of an external capsid and an internal core. The capsid is composed of 20 triangular surfaces or facets and 12 vertices (Home et al., J. Mol. Biol. 1:84-86 (1959)). The facets are comprised of hexons and the vertices are comprised of pentons. A fiber projects from each of the vertices. In addition to the hexons, pentons, and fibers, there are eight minor structural polypeptides, the exact positions of the majority of which are unclear. Adenovirus serotype classification is largely determined by epitopes on the hexon protein and the terminal knob portion of the fiber protein.

The viral core contains a linear, double-stranded DNA molecule of about 36 kb in length with inverted terminal repeats (ITRs), which have been noted to vary in length from about 100 bp to 160 bp in different isolates (Shenk, supra; Garon et al., Proc. Natl. Acad. Sci. USA, 69, 2391-2394, 1972; Wolfson et al., Proc. Natl. Acad. Sci. USA, 69, 3054-3057 (1972); Arrand et al, J. Mol. Biol., 128, 577-594 (1973); Steenberg et al, Nucleic Acids Res., 4, 4371-4389 (1977); Tooze, DNA Tumor Viruses (2nd ed., Cold Spring Harbor, N.Y.: Cold Spring Harbor Laboratory, 1981), pp. 943-1054). The ITRs harbor origins of DNA replication (Garon et al, supra; Wolfson et al, supra; Arrand et al., supra; Steenberg et al, supra).

The viral DNA is associated with four polypeptides, namely V, VII, mu, and terminal polypeptide (TP) (see, e.g., Shenk, supra). The 55 kd TP is covalently linked to the 5′ ends of the DNA via a dCMP (Rekosh et al, Cell 11:283-295, 1977 and Robinson et al., Virology 56:54-69, 1973). The other three polypeptides are noncovalently bound to the DNA and fold it in such a way as to fit it into the small volume of the capsid. The DNA appears to be packaged into a structure similar to cellular nucleosomes as seen from nuclease digestion patterns (Corden et al, Proc. Natl. Acad. Sci. USA, 73:401-404 (1976); Tate et al., Nucleic Acids Res. 6:2769-2785 (1979); Mirza et al., Biochim. Biophys. Acta 696:76-86 (1982)).

The cycle from cell infection to production of viral particles lasts 1-2 days and results in the production of up to 10,000 infectious particles per cell (Green et al., Virology 13:169-176 (1961)). The infection process of adenovirus is divided into early (E) and late (L) phases, which are separated by viral DNA replication, although some events that take place during the early phase also take place during the late phase and vice versa. Further subdivisions of the adenoviral genetic regions have been made to fully describe the temporal expression of viral genes.

During the early phase, viral mRNA is synthesized from both strands of the adenoviral DNA present in the cell nucleus. At least five regions, designated E1, including E1a and E1b, E2, E3, and E4, are transcribed (e.g., Shenk, supra; Lewis et al., Cell 7:141-151 (1976); Sharp et al., Virology 75:442-456 (1976); Sharp, “Adenovirus transcription,” in The Adenoviruses, Ginsberg, ed., Plenum Press, New York, N.Y., pp. 173-204 (1984)). Each region has at least one distinct promoter and is processed to generate multiple mRNA species.

31 The products of the early (E) regions (1) serve regulatory roles for the expression of other viral components, (2) are involved in the general shut-off of cellular DNA replication and protein synthesis, and (3) are required for viral DNA replication. The intricate series of events regulating early mRNA transcription begins with expression of certain immediate early regions, including E1A, L1, and the 13.5 kd gene (reviewed in Horwitz, supra). Expression of the delayed early regions E1B, E2A, E2B, E3 and E4 is dependent on the E1A gene products. Three promoters—the E2 promoter at 72 map units (“mu”), the protein IX promoter, and the IVa promoter—are enhanced by the onset of DNA replication, but are not dependent on it (Wilson et al., Virology, 94, 175-184, 1979). Their expression characterizes an intermediate phase of viral gene expression. The result of the cascade of early gene expression is the start of viral DNA replication.

As noted above, the adenoviral genome is a linear, double-stranded DNA of approximately 36 kb in length. This length can be incorporated into cosmid vectors, which typically can accommodate up to about 45-50 kb. The adenoviral DNA to be cloned into a cosmid vector can also include heterologous sequences, often of up to about 8 kb in length. Deletion of additional regions of the Ad7 genome can also increase the cloning capacity of the vector.

The Ad7 cosmid vectors of the invention are prepared by cloning an Ad7 adenoviral genome into a cosmid vector. Linear concatenated DNA substrates, suitable for packaging in vitro, are generated by ligating restriction fragments containing a cos sequence to each end of the genomic DNA. During packaging, the two flanking cos sequences are cleaved to generate a linear molecule with termini that are complementary to one another. The λ phage containing the cosmid DNA are used to infect susceptible bacterial cells. The complementary termini anneal to one another and are sealed by the host's DNA ligase, generating circular DNA molecules carrying a co1E1 plasmid replicon and a selectable marker. This system provides the ability to generate large quantities of Ad7 cosmid DNA. The Ad7 genome can then be released from the cos vector by digestion with an appropriate restriction endonuclease to release the adenoviral genome. The adenoviral genome is then used to infect host cells to produce adenoviral particles.

The Ad7 cosmids often contain deletions in particular regions of the genome to prevent replication and/or to accommodate heterologous nucleic acid sequence encoding one or more polypeptides to be expressed. These polypeptides are typically contained in expression cassettes. As appreciated by one of skill in the art, the expression cassette can include control sequences, e.g., promoters, enhancers operably linked to the heterologous nucleic acid sequence. Control sequences can comprise Ad sequences normally associated with wild-type Ad genome, e.g., the adenovirus major late promoter, or heterologous control sequences can be employed. Useful heterologous promoter sequences include those derived from sequences encoding mammalian genes or viral genes, e.g., a CMV promoter such as the CMV immediate early promoter region, an SV40 promoter, a mouse mammary tumor virus LTR, a herpes simplex virus promoter, a Rous sarcoma virus promoter, synthetic promoters, hybrid promoters, and tissue-specific promoters and the like.

If multiple heterologous nucleic acid sequences are to be included in the Ad7 vector, for example, if a second heterologous nucleic acid sequence is to be expressed in the Ad7 construct, the second sequence can be included with the first sequence in a single expression cassette or can be incorporated in a second expression cassette that can be positioned in another region of the adenoviral genome. Thus, the vectors can accommodate mutliple sequences to be expressed.

The Ad7 vectors of the invention are often replication-deficient, i.e., the adenoviral DNA cannot replicate in the absence of products provided in trans. The replication-deficient Ad7 vectors of the invention can be deficient in a number of regions, such as any region(s) in the early or late regions required for viral replication. Typically, the vector lacks a functional early region gene, particularly a gene in the E1 region, for example, either the E1A region alone or both the E1A and E1B regions. As appreciated by one of skill in the art, the deficiency can be introduced by varioius mutations including single base substitutions, small deletions, insertions and the like, but is most often achieved by deletion of part or all of the particular region.

Often, the Ad7 vector is deficient in at least one additional region, e.g., a vector that lacks a functional E1A region can also lack a functional region of another gene, for example E1B, E2, including E2A and/or E2B, E3, and E4. Deficiencies, typically deletions, can also be introduced into late regions of the adenoviral genome. For example, additional deletions in the non-essential E3 region are frequently introduced to increase the packaging capacity of such vectors. Replication defective adenoviral vectors may also contain mutations or deletions so as to substantially eliminate protein IX function.

As is apparent to one of ordinary skill in the art, the Ad7 cosmid expression constructs comprising one or more polypeptides to be expressed can be generated in a variety of ways. Typically, the cosmid comprising the completed adenoviral genome is created by using two, often three, shuttle vectors to introduce a specific modification into a portion of the Ad7 genome and then assembling the regions of the genome contained in the shuttle vectors to create a complete adenoviral genome subcloned into a cosmid vector. At least one, often two, of the shuttle vectors typically comprises a cos sequence.

For example, an initial step in vector construction can be the introduction of a deletion or modification to a particular region of the adenoviral genome e.g., a deletion in all or part of E1 and/or E3. This modification is performed using a plasmid shuttle vector comprising a portion of the Ad7 genome that includes the E1 or E3 region. Deletion can be accomplished using standard molecular biological techniques. The deleted region is often also used as a cloning site to introduce an expression cassette. The altered DNA (containing the deletion, modification, or expression cassette) can be ligated to one or more fragments of adenoviral DNA containing the remainder of the genome. The complete genome including fragments that contain the cos vector sequences is then ligated to reconstitute a cosmid vector. The ligation steps can be performed consecutively or concurrently.

Following creation of the cosmid comprising the Ad7 expression construct, the cosmid DNA is packaged into X phage and used to infect host cells to obtain colonies. Verification of the identity of the cosmid vectors is typically performed by restriction endonuclease digestion and sequence analysis. The colonies can then be used to produce large quantities of the Ad7 cosmid DNA.

As appreciated by one of skill in the art, the shuttle constructs generated to create a cosmid Ad7 vector system, can also be used to create adenoviruses through homologous recombination. In some applications, the cosmid-based shuttle vectors may provide a more efficient means of generating the adenovirus.

Generation of Ad7 Adenoviruses

The Ad7 virus is created by releasing the Ad7 genome from the cosmid by restriction enzyme digestion and transfecting a packaging or host cell line to obtain infectious adenoviral products. In the case of a replication defective Ad7 construct, the host cell line typically includes a complementing activity to allow the Ad7 to replicate. Preferably, the complementary cell line specifically complements for those functions that are missing from the replication-deficient Ad7 vector. For example, an Ad7 construct that is deleted or lacking a functional E1 regions is introduced into a host cell line that provides the E1 function in trans. Such a cell line also preferably contains the complementing gene(s) in a nonoverlapping fashion so as to minimize, if not eliminate, the possibility of vector recombination yielding a replication-competent adenoviral vector. An example of an appropriate cell line for the propagation of replication-defective Ad7 adenoviruses is the A232 cell line, also referred to as 293-ORF6, which provides the E1 function in trans to allow replication of an E1-deleted Ad7 (see, e.g., Brough et al., J. Virol. 70:6497-6501, 1996). Permissive conditions for Ad replication and the like are known and described in the scientific and patent literature, see, e.g.,U.S. Pat. Nos. 5,837,511; 5,851,806; and 5,994,106.

The level of expression of the expression products encoded by the Ad7 vector is typically analyzed by any number of methods including ELISA or western blotting. The resulting Ad7 virus can then be used as a vaccine or gene delivery vehicle for gene transfer.

The Ad7 virus vaccines can be tested using a number of different assays to assess induction of an immune response to the gene encoded by the expression cassette. These include both cytotoxic and helper T cell responses as well as humoral immune responses. Induction of cytotoxic T lymphocytes can, for example, be assayed using chromium release assays and assessment of interferon gamma secretion of PBMCs using an ELISPOT assay. Induction of a T-helper lymphocyte immune response can be measured using a proliferation assay based on thymidine incorporation. Humoral immune responses can be assayed, for example, by ELISA and neutralization assays. Such assays are described, for example, in CURRENT PROTOCOLS IN IMMUNOLOGY, Wiley/Greene, NY, (1998).

Replication-Defective Ad7 HIV Vaccines

In one aspect, the invention provides replication defective HIV Ad7 vectors. Such vectors can be used as a vaccine to prevent or treat HIV infection. As appreciated by one of skill in the art, a number of highly antigenic epitopes for provoking an immune response selective for HIV-1 are known. HIV-specific epitopes fall into two major categories, structural and non-structural proteins. Epitopes can be selected from either or both groups of proteins. Structural proteins are a physical part of the virion. Non-structural proteins are regulatory proteins. The envelope is a preferred source of epitopes and the precursor envelope protein, gp160, and its components, the extracellular gp120 and the transmembrane protein gp41, are sources of immunoprotective epitopes. Both B and T cell epitopes have been described in the literature and can be used. Epitopes selected from the V3 loop of the HIV envelope proteins, for example, have been of preferred use. In addition other structural proteins have been reported to contain immunoprotective epitopes including proteins encoded by the gag and pol genes. Non-structural genes include the rev, tat, nef, vif, and vpr genes. Any number of these genes can be included in an Ad7 HIV vaccine of the invention.

Preferably, the HIV Ad7 vaccines are replication-defective for safety considerations. Such vaccines can be used alone or in conjunction with other HIV vaccines, e.g., AdS or Ad2 HIV vaccines, as explained below.

The immunogenicity of the HIV Ad7 vaccines can be tested by measuring the induction of T cell and B cell responses using such assays as those described above.

Formulation and Administration of Pharmaceuticals

As discussed above, the present invention also provides recombinant Ad7 vectors for use in vaccines and as gene transfer vectors. The adenovirus prepared as described herein can be formulated for administration to a mammalian organism in accordance with techniques well known in the art. The viruses can be administered in conventional solutions such as sterile saline and can incorporate one or more pharmaceutically acceptable carriers or excipient to form a pharmacological composition. The pharmaceutical composition can further comprise other active agents, including other recombinant viruses, plasmids, naked DNA or other agents.

The compositions for administration typically comprise a buffered solution comprising adenovirus in a pharmaceutically acceptable carrier, e.g., an aqueous carrier. A variety of carriers can be used, such as buffered saline, water and the like. These solutions are generally sterile and free of undesirable matter. These compositions can be sterilized by conventional, well known sterilization techniques, or can be sterile filtered. The compositions may contain pharmaceutically acceptable auxiliary substances as required to approximate physiological conditions such as pH adjusting and buffering agents, tonicity adjusting agents and the like, for example, sodium acetate, sodium chloride, potassium chloride, calcium chloride, sodium lactate and the like.

Pharmaceutically acceptable carriers can contain a physiologically acceptable compound that acts, e.g., to stabilize the composition or to increase or decrease the absorption of the virus and/or pharmaceutical composition. Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of any co-administered agents, or excipient, or other stabilizers and/or buffers. Detergents can also be used to stabilize the composition or to increase or decrease absorption.

The Ad7 constructs can also be administered in a lipid formulation, more particularly either complexed with liposomes or to lipid/nucliec acid complexes (e.g., WO 93/24640; U.S. Pat. No. 5,279,833, and WO 91/06309) or encapsulated in liposomes, as in immunoliposomes directed to specific tumor markers.

The Ad7 constructs can also be administered orally as enteric coated capsules as previously described, in order to bypass the upper respiratory tract and replicate in the gut: see, e.g., Tacket et al., Vaccine 10:673-676, 1992; Horwitz, in Fields et al, eds., Fields Virology, third edition, vol 2, pp. 2149-2171, 1996; Takafuji et al., J. Infec. Dis. 140:48-53, 1979; and Top et al., J. Infec. Dis. 124:155-160,1971.

One skilled in the art would appreciate that the choice of a pharmaceutically acceptable carrier, including a physiologically acceptable compound depends, e.g. on the route of administration of the adenoviral preparation and on the particular physio-chemical characteristics of any co-administered agent.

In some embodiments of the invention, the targeted complexes of the invention are administered ex vivo to cells or tissues explanted from a patient, then returned to the patient. Examples of ex vivo administration of therapeutic gene constructs include Arteaga et al., Cancer Research 56(5):1098-1103 (1996); Nolta et al. Proc. Nat'l. Acad. Sci. USA 93(6):2414-9 (1996); Koc et al., Seminars in Oncology 23 (1):46-65 (1996); Raper et al., Annals of Surgery 223(2):116-26 (1996); Dalesandro et al., J. Thorac. Cardi. Surg., 11(2):416-22 (1996); and Makarov et al., Proc. Nat'l. Acad. Sci. USA 93(1):402-6 (1996).

Administration

The compositions can be administered alone, or can be co-administered or sequentially administered with other immunological, antigenic, vaccine, or therapeutic compositions. Such compositions can include other agents to potentiate or broaden the immune response, e.g., IL-2 or other cytokines which can be administered at specified intervals of time, or continuously administered (see, e.g., Smith et al., N Engl J Med 1997 April 24;336(17):1260-1; and Smith, Cancer J Sci Am. 1997 Dec;3 Suppl 1:S137-40). The vaccines or vectors can also be administered in conjunction with other vaccines or vectors. For example, an adenovirus type 7 of the invention can be administered either before or after administration of an adenovirus of a different serotype. An Ad7 preparation may also be used, for example, for priming in a vaccine regimen using an additional vaccine agent.

The adenoviral formulations can be delivered systemically, regionally, or locally. Regional administration refers to administration into a specific anatomical space, such as intraperitoneal, intrathecal, subdural, or to a specific organ, and the like. Local administration refers to administration of a composition into a limited, or circumscribed, anatomic space such as an intratumor injection into a tumor mass, subcutaneous injections, intramuscular injections, and the like. One of skill appreciates that local administration or regional administration can also result in entry of the viral preparation into the circulatory system. Typical delivery routes include parenteral administration, e.g., intradermal, intramuscular or subcutaneous routes. Other routes include oral administration, intranasal, and intravaginal routes. For delivery of adenovirus, administration can often be performed via inhalation.

The vectors of the current invention, alone or in combination with other suitable components can be made into aerosol formulations to be administered via inhalation. These aerosol formulations can, for example, be placed into pressurized, pharmaceutically acceptable propellants, such as dichlorodifluoromethane, nitrogen and the like. They can also be formulated as pharmaceuticals for non-pressurized preparations such as in a nebulizer or an atomizer. Typically, such administration is in an aqueous pharmacologically acceptable buffer as described above. Delivery to the lung can also be accomplished, for example, using a bronchoscope.

The vaccines can also be formulated for administration via the nasal passages. Formulations suitable for nasal administration, wherein the carrier is a solid, include a coarse powder having a particle size, for example, in the range of about 10 to about 500 microns which is administered in the manner in which snuff is taken, i.e., by rapid inhalation through the nasal passage from a container of the powder held close up to the nose. Suitable formulations wherein the carrier is a liquid for administration as, for example, nasal spray, nasal drops, or by aerosol administration by nebulizer, include aqueous or oily solutions of the active ingredient. For further discussions of nasal administration of AIDS-related vaccines, references are made to the following patents, U.S. Pat. No. 5,846,978, 5,663,169, 5,578,597, 5,502,060, 5,476,874, 5,413,999, 5,308,854, 5,192,668, and 5,187,074.

Formulations for oral administration can consist of liquid solutions, such as an effective amount of the pharmaceutical dissolved in ingestible diluents, such as water, saline, orange juice, and the like; capsules, or tablets containing a predetermined amount of the active ingredient; suspensions in an appropriate liquid; and suitable emulsions.

Additionally, the vectors can be made into suppositories by mixing with a variety of bases such as emulsifying bases or water-soluble bases. Formulations suitable for vaginal administration can be presented as pessaries, tampons, creams, gels, pastes, foams, or spray formulas.

The pharmaceutical formulation of the invention can be administered in a variety of unit dosage forms, depending upon the particular condition or disease, the general medical condition of each patient, the method of administration and the like. In one embodiment for a vaccine, the concentration of adenovirus in the pharmaceutically acceptable excipient can be from about 10³ to about 10¹⁸ or between about 10⁵ or 10¹⁵ or between about 10⁶ to about 10¹³ particles per mL in an aqueous solution. Details on dosages are well described in the scientific and patent literatures, see, e.g., the latest edition of Remington's Pharmaceutical Sciences; Sterman et al., Hum. Gene Ther 9:1083-1092, 1998; and Smith et al., Hum. Gene Ther. 8:943-954, 1997.

The amount and concentration of virus and the formulation of a given dose, or a “therapeutically effective” dose is determined by the clinician as discussed herein. A therapeutically effective dose of a vaccine is an amount of adenovirus that will stimulate an immune response to the protein(s) encoded by the heterologous nucleic acid included in the viral vector. For gene therapy, a therapeutically effective dose can be an amount sufficient to reduce symptoms of a disease.

The dosage schedule, i.e., the dosing regimen, will depend upon a variety of factors, e.g., the stage and severity of the disease or condition to be treated, and the general state of the patient's health, physical status, age and the like. The state of the art allows the clinician to determine the dosage regimen for each individual patient. Adenoviruses have been safely used for many years for human vaccines; see, e.g., Franklin et al, supra; Jag-Ahinade et al., J. Virol., 57:267, 1986; Ballay et al, EMBO J. 4:3861, 1985; PCT publication WO 94/17832. They have also been used in humans as in vivo gene delivery vehicles (e.g., Graham & Prevec in New Approaches to Immunological Problems, Ellis led), Butter worth-Heinemann, Boston, Mass., pp. 363-390, 1992; Rago et al, Nature 361:647-650, 1993; Kozarsky, Curr. Opin. Genet. Dev. 3:499-503, 1993; and U.S. Pat. Nos. 5,981,225, and 5,837,511). These illustrative examples can also be used as guidance to determine the dosage regimen when practicing the methods of the invention.

Single or multiple administrations of adenoviral formulations can be administered, depending on the dosage and frequency as required and tolerated by the patient. Thus, one typical dosage for regional, e.g., IP administrations is between about 0.5 to about 50 mL of a formulation with about 10¹³ viral particles per mL. In an alternative embodiment, dosages from about 5 mL to about 20 mL are used of a formulation with about 10⁹ viral particles per mL. Lower dosages can be used, such as between about 1 mL to about 5 mL of a formulation with about 10⁶ viral particles per mL.

For vaccines, wild-type Ad4 and Ad7 vaccines can be administered at a range of dosages. For examples, 10⁷ to 10⁹50% tissue culture infective doses (or plaque forming units) can be administered, typically an oral dosages is about 10⁷ 50% tissue culture infective doses: see, e.g., Top et al., J. Infec. Dis. 124:155-160, 1971; Takafuji et al., J. Infec. Dis. 140:48-53, 1979, or 10⁷ plaque forming units: see, e.g Tacket et al., Vaccine 10:673-676, 1992. Intranasal administration of adenovirus vaccines is often in dosages of from about 10⁴ to 10⁷ plaque forming units. Typically, dosages of 10⁴ or 10⁵ plaque forming units: see, e.g., Smith et al., J. Infec. Dis. 122:239-248, 1970 are administered. The exact concentration of virus, the amount of formulation, and the frequency of administration can also be adjusted depending on the levels of in vivo, e.g., in situ transgene expression and vector retention after an initial administration.

Kits

The invention also provides kits that contain the vectors, vector systems or pharmaceutical compositions of the invention. The kits can, for example, also contain replication-competent cells. The kits can includes instructional material teaching methodologies for generating adenoviruses using the kits and, for pharmaceutical compositions, can include instruction for indication of dosages, routes and methods of administration and the like.

### EXAMPLES

### Example 1

### Construction of Ad7 Cosmid Vectors

Generation of Shuttle Plasmids

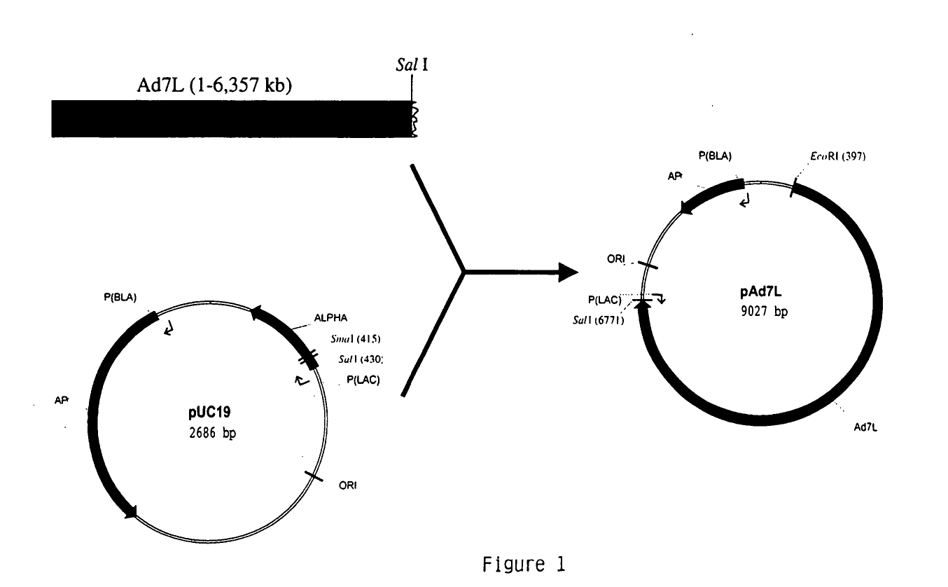

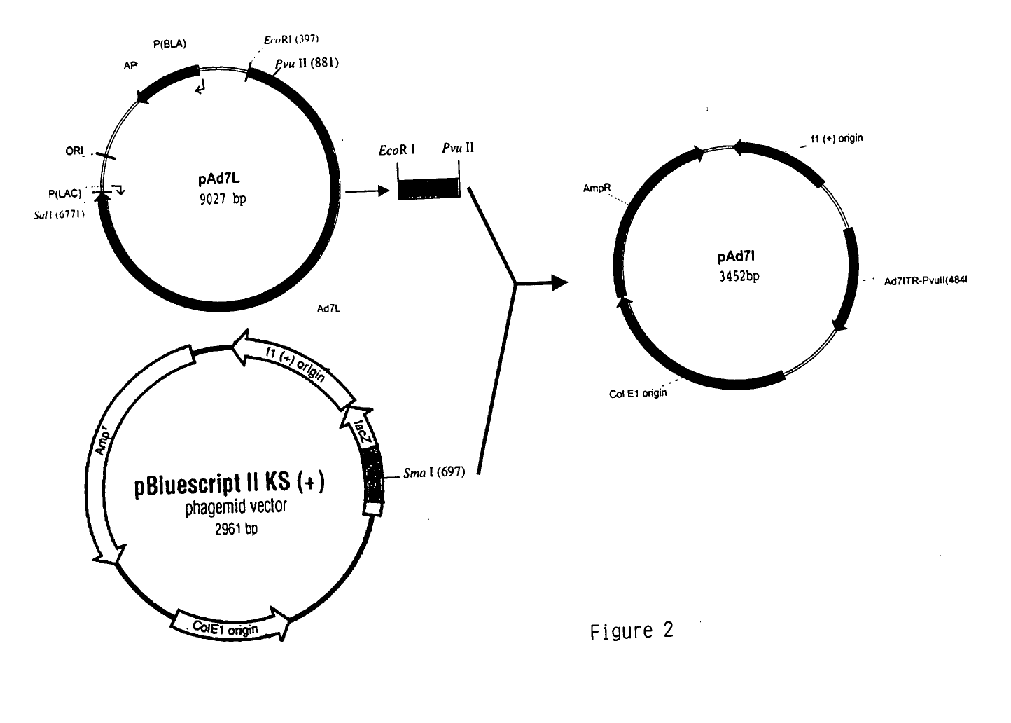

The left SalI fragment (i.e., base pairs 1-6357) of adenovirus serotype 7 was first cloned into pUC19 at the SmaI and SalI sites, thereby generating pAd7L (FIG. 1). The EcoRI-PvuII digested, filled-in fragment from pAd7L containing the left end (base pairs 1-466) of Ad7 was inserted at the SmaI site of pBluescript II KS(+), yielding pAd7I (FIG. 2).

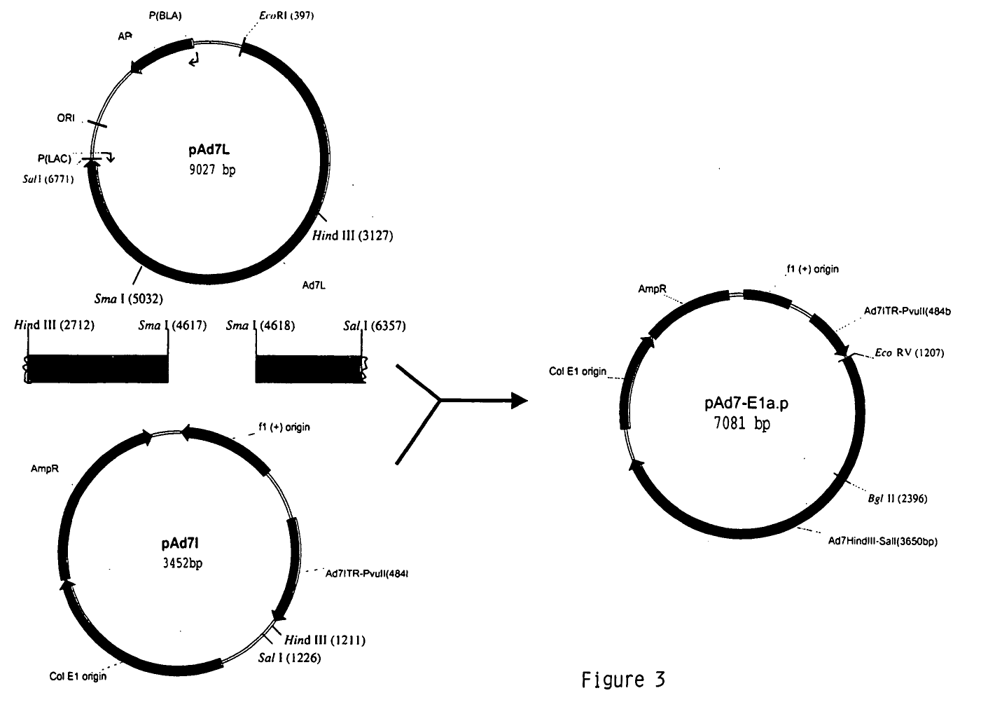

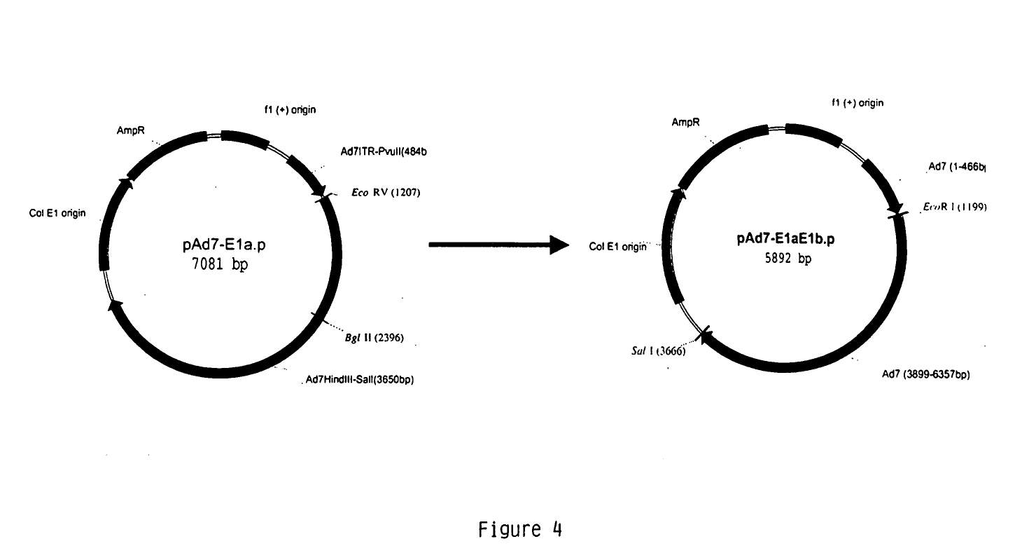

The E1 a-deleted plasmid, pAd7ΔE1a.p, was constructed by cloning the HindIII-SmaI (base pairs 2,712 to 4,617 and SmaI-SalI (base pairs 4,618 to 6,357) fragments from pAd7L in tandem downstream of the EcoRI-PvuII fragment in pAd7I into HindIII, SalI-digested pAd7I (FIG. 3). The EcoRV-BglII fragment from pAd7ΔE1a.p containing the E1b sequences (base pairs 2,712 to 3,897) was further deleted to generate pAd7ΔE1aE1b.p (FIG. 4). Deletion of the E1b sequences also deleted the coding region for protein IX, which is adjacent to E1b. Thus, this plasmid is also deleted in protein IX.

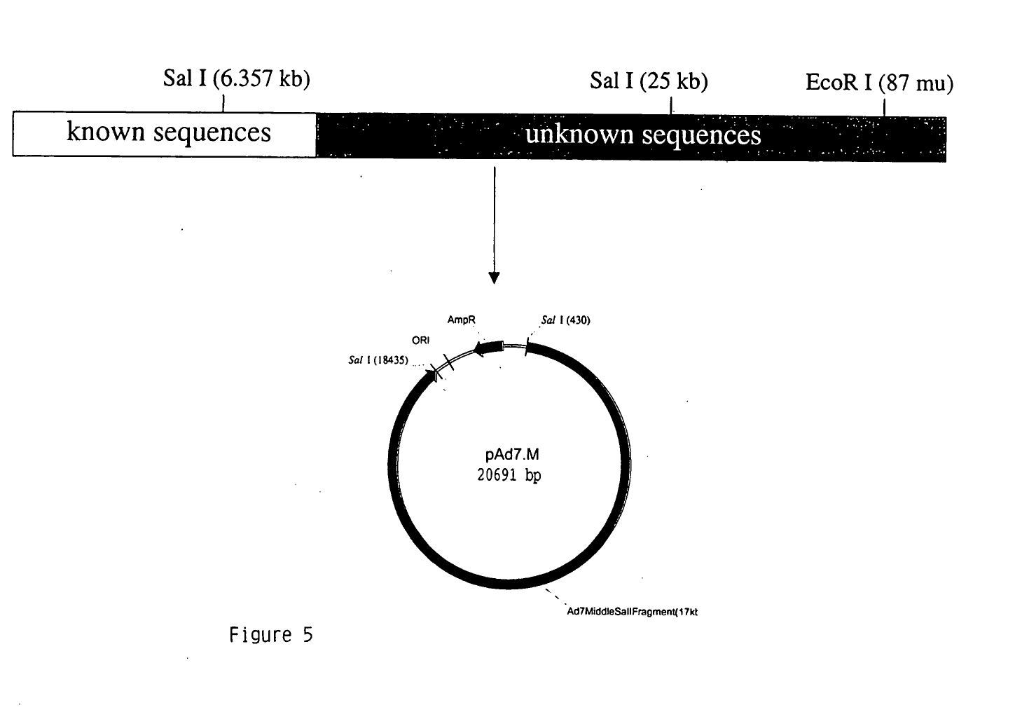

The plasmid pAd7M was generated by insertion of the (about) 20 kb SalI fragment from Ad7 into pUC19 (FIG. 5).

Preparation of the Cosmid Vector

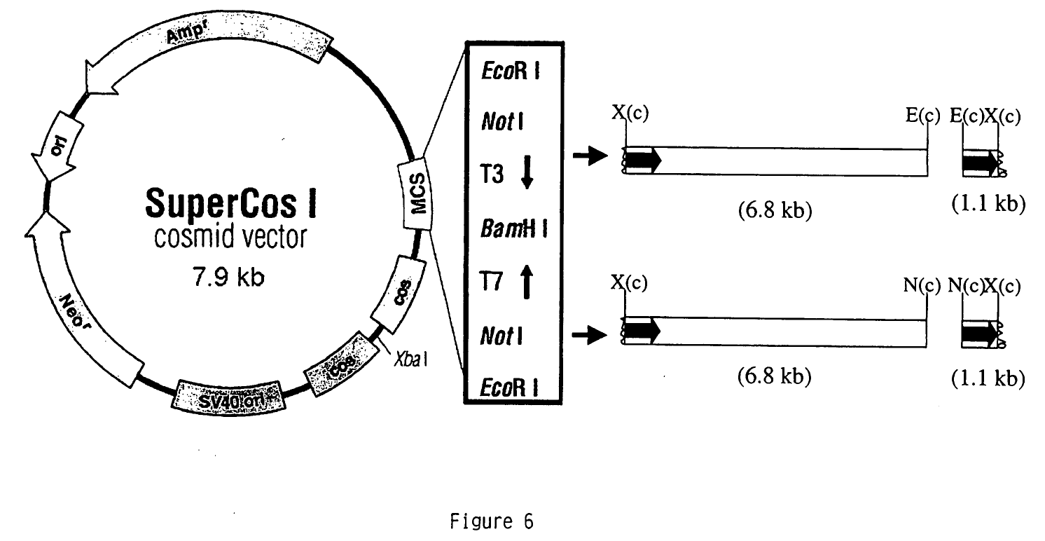

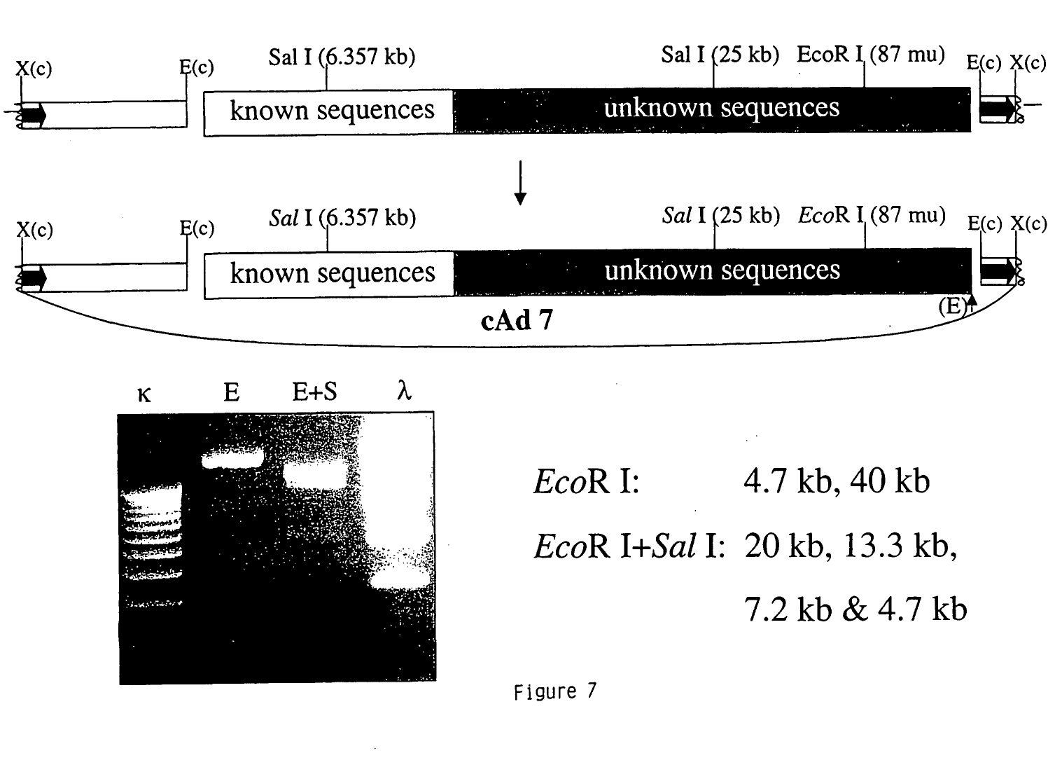

The SuperCos 1 cosmid vector (Stratagene) was first digested with either EcoRI or NotI, filled-in, digested with XbaI and dephosphorylated (FIG. 6). Digestion yields two cos arms, a 6.8 kb arm, the large cos arm; a 1.1 kb arm, the small cos arm. The cos vector can accommodate DNA inserts ranging in size from about 30 to about 42 kb. The cosmid cAd7 was generated by ligating the wild-type Ad7 genome into the EcoRI-digested cos arms (FIG. 7). The ligated DNA was packaged into phage X using Gigapack III XL packaging extract according to the manufacturer (Stratagene). The phage were then used to infect the Escherichia coli host strain XL-Blue MR. Colonies were selected using ampicillin and analyzed by restriction endonuclease digestion. The identity of positive clones was confirmed by sequencing.

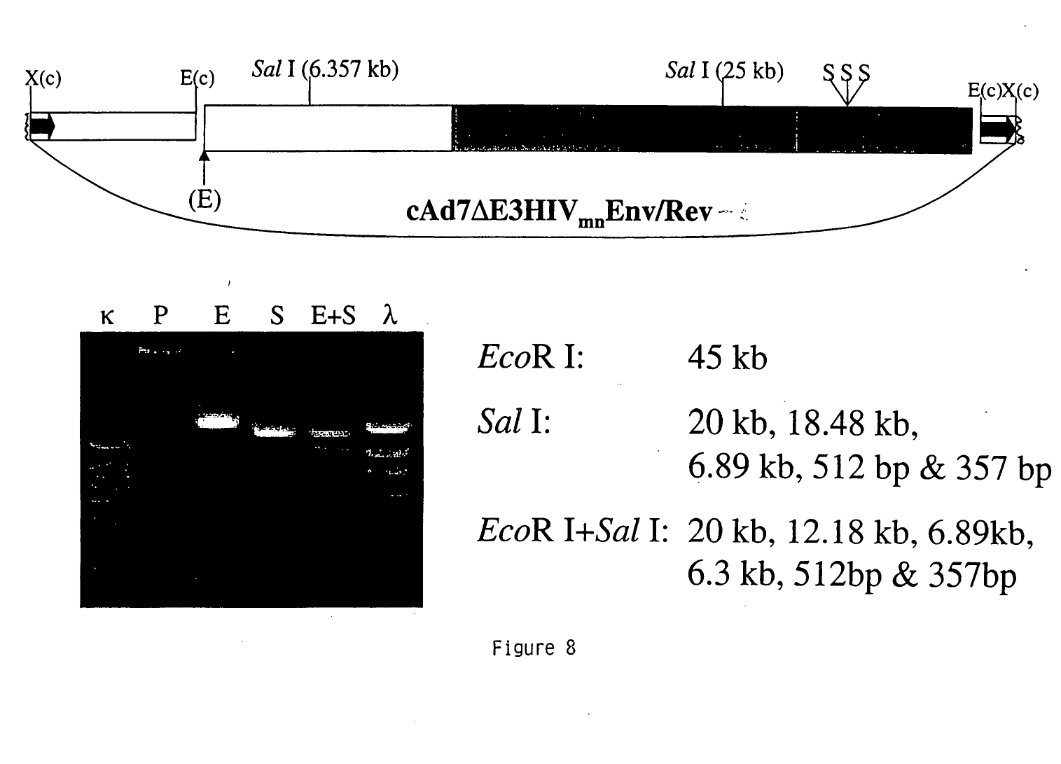

The cosmid cAd7ΔE3HIV_{MN}Env/rev-E was generated by ligating the isolated Ad7ΔE3HIV_{MN}Env/Rev genome DNA to the EcoRI-treated cos arms and packaging the ligation product (FIG. 8). Colonies were analyzed by restriction analysis and confirmed by sequencing. The cosmid cAd7ΔE3HIV_{MN}Env/rev-N was generated by ligating the Ad7ΔE3HIV_{MN}Env/Rev genome DNA into the two NotI-treated cos arms. The ligation product was packaged and used to infect the host cells. Ampicillin resistant colonies were selected and confirmed by restriction analysis and sequencing.

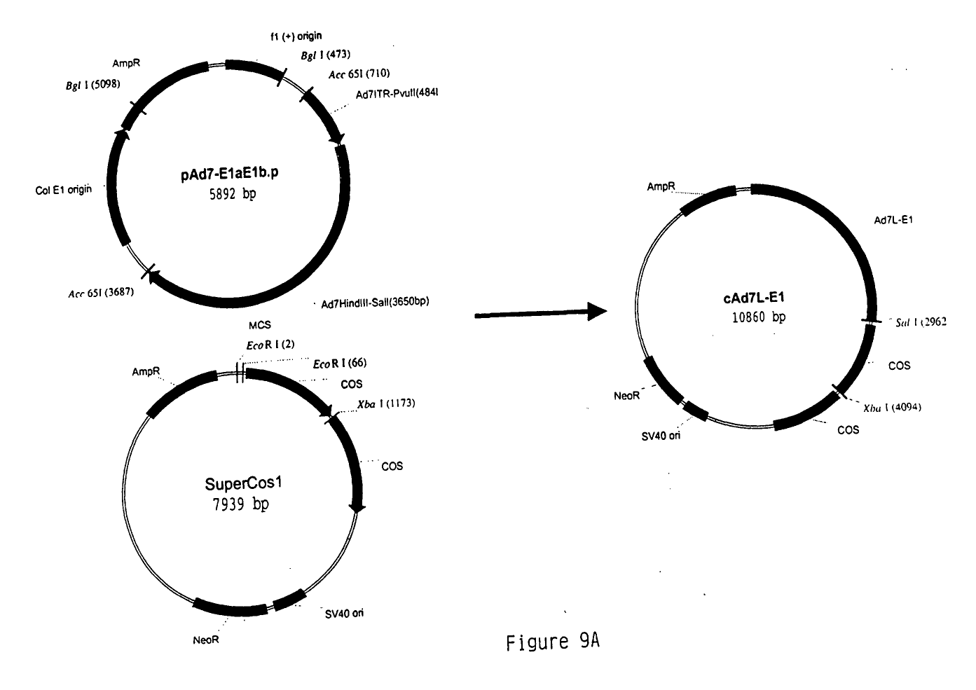

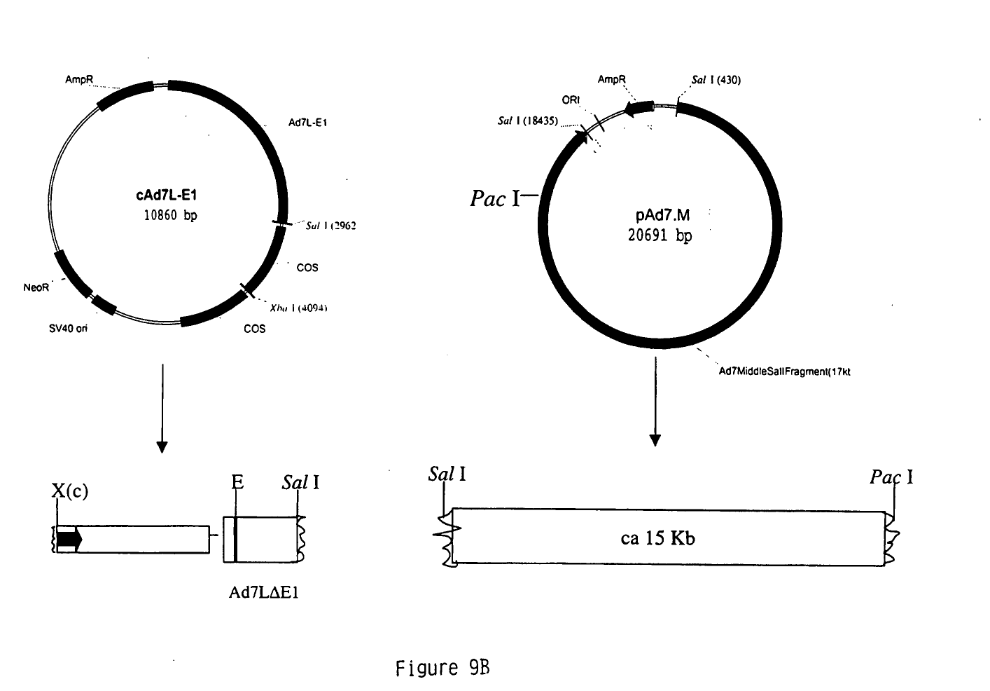

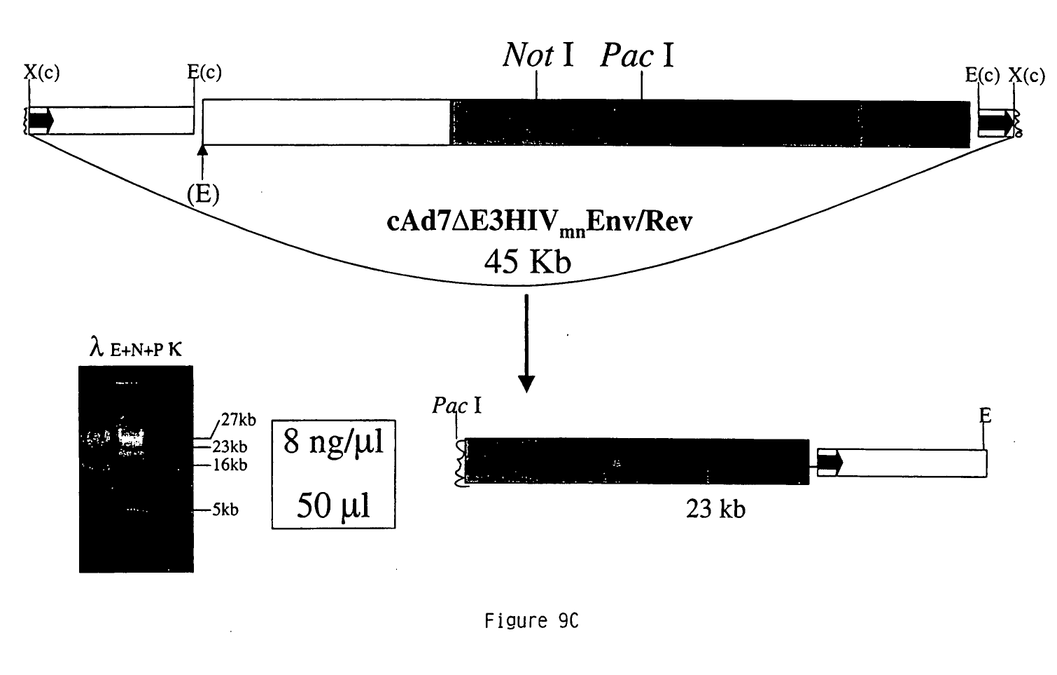

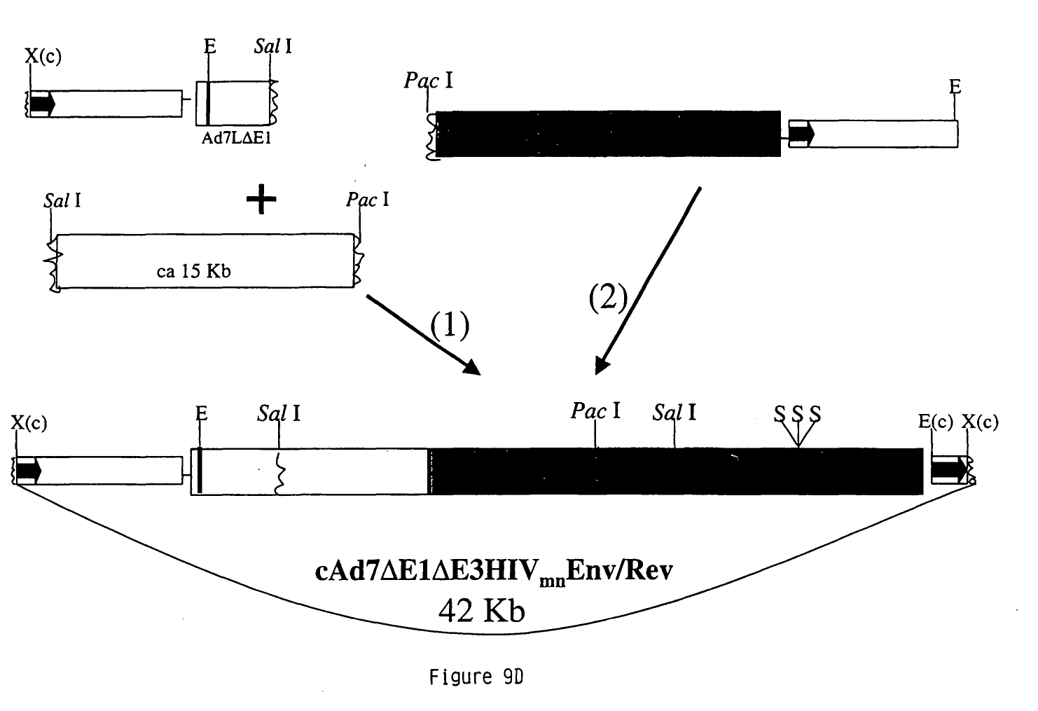

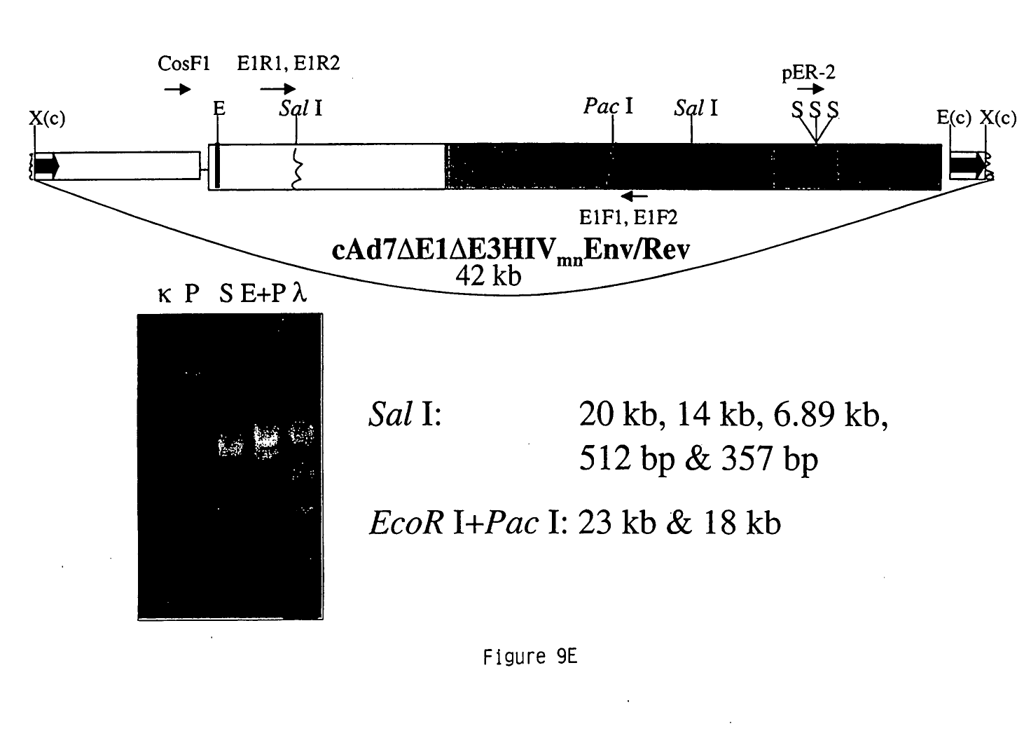

The cosmid cAd7ΔE1ΔE3HIV_{MN}Env/rev was obtained as follows (FIGS. 9A-9E). An AccI-digested, filled in fragment from pAd7ΔE1aE1b.p, which lacks the E1 gene, was inserted into an EcoRI-digested Super Cos 1 cosmid vector that had been blunt ended, thereby yielding cAd7LΔE1 (FIG. 9A). The cosmid cAd7ΔE1ΔE3HIV_{MN}Env/rev was created by a three-way ligation of the following fragments: the left XbaI-SalI fragment from cAd7LΔE1, which contains the large cos arm and left SalI fragment of Ad7 lacking the E1 gene (FIG. 9B); the middle SalI-PacI fragment of pAd7 (FIG. 9B); and the right PacI-EcoRI fragment from cAd7ΔE3HIV_{MN}Env/rev, which contains the small cos arm and the sequence from the PacI site located at about 20 kb to the right end of the Ad7 genome (FIG. 9C). The ligation reaction was packaged, used to infect host cells, and colonies selected. The identity of the positive colonies containing the correctly oriented product was determined by restriction enzyme analysis and sequencing.

New Vectors for the Construction of Single or Double Recombinant Adenoviruses.

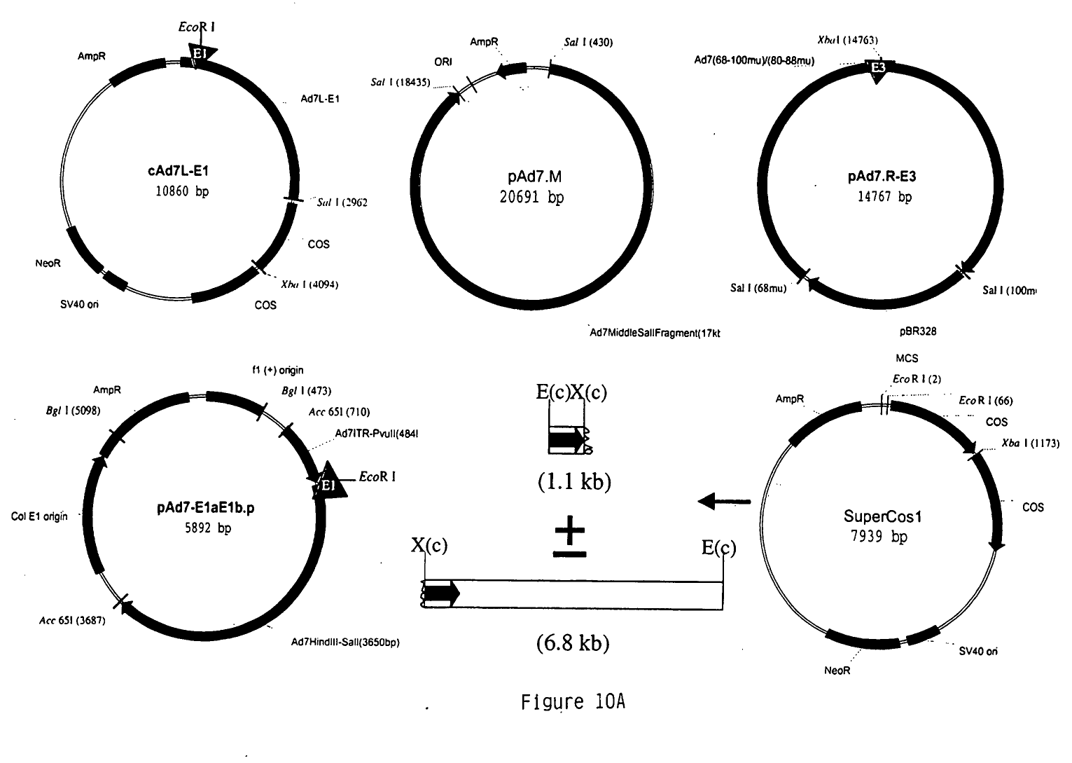

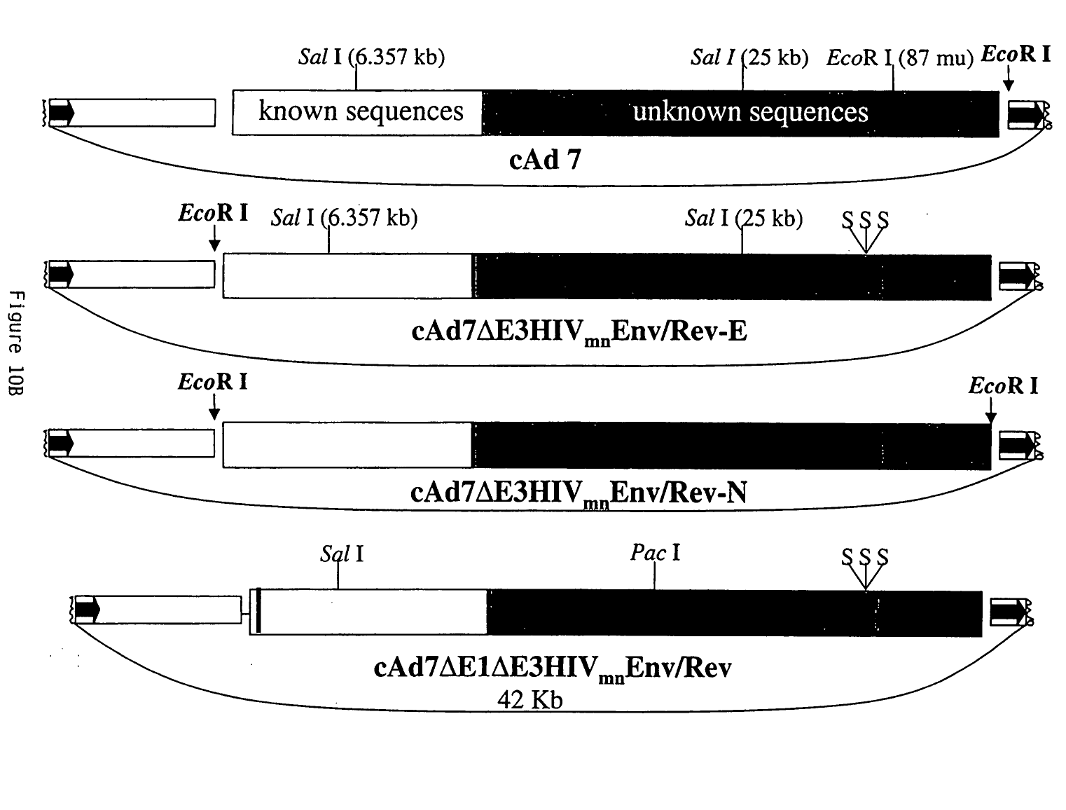

A new Ad7 cosmid system was created that consist of the left and right shuttle Ad7 vectors, the large middle Ad7-containing plasmid and the three recombinant adenoviral cosmids (FIGS. 10A and 10B). A gene of interest can be substituted for the E1 and/or E3 gene regions and then be cloned into the left (cAd7LΔE1 or pAd7ΔE1aE1b.p) and/or right shuttle vector (pAd7RΔE3) and then be ligated together with the large middle SalI fragment from pAd7M and the cos arms. If cAd7LΔE1 is used as the shuttle vector, only the small cos arm is required for packaging. By contrast, the big cos arm is also required for packaging if pAd7ΔE1aE1b.p is used as the shuttle vector. Thus, the shuttle vectors and recombinant adenoviral cosmids can be used to construct new recombinant adenoviral cosmids as illustrated by the generation of cAd7ΔE1ΔE3HIV_{MN}Env/rev described above. These new recombinant adenoviral cosmids can then be easily used for cloning purposes and avoids the necessity of isolating DNA directly from the infected cells that is typically required in analyzing adenovirus generated by homologous recombination.

### Example 2

### Analysis of Expression Products of Replication Defective Recombinant Adenoviruses

A replication defective recombinant Ad7 virus generated as outlined above can be analyzed for expression by digesting the recombinant cosmid to release the adenoviral genome and transfecting the genome into a host cell line, such as the 293-ORF6 cell line (Genvec). The host cell line provides E1 in trans to allow replication of the virus. The level of viral expression is analyzed, for example, by western blot analysis to detect the expressed protein.

Lysates of cells transfected with the Ad7 recombinants were analyzed by western blotting using the monoclonal antibody 2F5, which is specific for an epitope in the HIV gp41. Lysates of cells infected with Ad7 recombinants were immune precipitated with the 2F5 antibody. The following cells were analyzed: Ad7ΔE3HIVMNenv/rev-infected 293 cells; Ad7ΔE1 ΔE3HIVMNenv/rev-infected 293-ORF6 cells; uninfected 293-ORF6 cells; uninfected A549 cells; Ad7ΔE3HIVMNenv/rev-infected A549 cells; and Ad7ΔE1ΔE3HIVMNenv/rev-infected A549 cells. Migration of proteins with the expected sizes of 160 kDa and 41 kDa was observed in the Ad7ΔE3HIVMNenv/rev-infected 293 cells and Ad7ΔE1 ΔE3HIVMNenv/rev-infected 293-ORF6 cells. Protein expression was also observed in the Ad7-infected A549 cells, although the replication-defective Ad7ΔE1ΔE3HIV_{MN}Env/rev only expressed a low level of HIV envelope proteins in A549 cells (which lack the E1 gene) compared to the replication competent Ad7ΔE3HIV_{MN}Env/rev.

Syncytium formation of CD4⁺ T-cells was also examined as a further indication of HIV envelope expression and processing. The envelope gp160 precursor envelope protein of HIV must be cleaved to gp120 and gp41 by a trypsin-like host cell protease in order for the virus to bind the CD4 receptor and for virus-cell fusion to occur. A cell fusion assay was carried out to evaluate envelope processing. 293-ORF6 cells were infected with Ad7ΔE1ΔE3HIV_{MN}Env/rev. Four hours later the cells were overlayed with CD4⁺ CEMX174 cells. The cultures were examined 48 to 72 hours later for presence of syncytia. Syncytia were readily observed following infection with Ad7ΔE1ΔE3HIV_{MN}Env/rev, however, no syncytia could be detected when 293-ORF6 cells were first infected with wild-type Ad7 or with uninfected cells. This experiment indicates that the HIV envelope protein is expressed and properly processed in order for CD4 binding and cell fusion to occur.

### Example 3

### Immunogenicity of Ad7ΔE1ΔE3HIV_{MN}Env/rev

The replication defective Ad7ΔE1ΔE3HIV_{MN}Env/rev adenovirus is tested for immunogenicity in chimpanzees. The results show that Ad7ΔE1ΔE3HIV_{MN}Env/rev elicits an immune response. The vaccine can also further be tested in conjunction with Ad5-based vectors.

Chimpanzees are used for these experiments because adenoviruses are severely host-range restricted and chimpanzees provide a good model for testing a vaccine regimen that includes a replication-competent Ad virus.

Chimpanzees with minimal Ad5- and Ad7-cross-reactive antibodies are be selected for this experiment. Chimpanzees are inmmunized according to the schedule in Table 1. Ad-recombinants diluted in PBS are administered drop-wise into the nostrils; 1 ml total volume, 500 μl per each nostril. Antibiotics are administered for a total of 11 days, beginning 3 days prior to inoculation. This is now a routine procedure, due to a previous death of a chimpanzee from bacterial pneumonia who was found retrospectively to have an elevated temperature and other clinical abnormalities on the day of Ad-recombinant administration. The death was attributed to S. Pneumoniae. There was no evidence of infectious adenovirus in blood or tissues of the chimpanzee upon autopsy (Natuk et al., AIDS Res. Hum. Retroviruses 9:395, 1993).

TABLE 1

| Chim- | | | Week |
|---|---|---|---|
| panzees | Week 0 (IN) | Week 12 (IN) | 36 (IM) |
| 3 | Ad5-ΔE3-HIV gp160 | Ad7-ΔE3-HIV gp160 | native |
| | 10⁷ pfu (replication | 10⁷ pfu (replication | gp 120 |
| | competent) | competent) | |
| 2 | Ad5-ΔE3-HIV gp160 | Ad7-ΔE3-HIV gp160 | native |
| | 10⁸ pfu | 10⁸ pfu | gp 120 |
| 2 | Ad5-ΔE1-ΔE3-HIV | Ad7-E1-ΔE3-HIV gp160 | native |
| | gp160 10⁸ pfu | 10⁸ pfu (replication | gp 120 |
| | (replication defective) | defective) | |
| 3 | Ad5-ΔE1-ΔE3-HIV | Ad7-ΔE1-ΔE3-HIV gp160 | native |
| | gp160 10⁹ pfu | 10⁹ pfu | gp 120 |

The protein subunit (50 μg) is administered in adjuvant. Blood, secretory samples, and stool specimens are collected at various intervals of one, two, or four weeks up to a year.

Various immune responses are measured including both cellular and humoral responses. Cellular immune responses against both adenovirus and the inserted HIV env and rev gene products are assayed by: 1) assay of cytotoxic T lymphocytes, using the chromium release assay and/or 2) assay of interferon gamma secretion of PBMCs by ELISPOT assay; and 3). proliferation assay of T-helper lymphocytes by thymidine incorporation Humoral immune responses against both adenovirus and the inserted HIV env and rev gene products are assayed by: 1). ELISA assay for HIV gp120 binding antibodies in serum and mucosal secretions; 2) Microtiter neutralization assay for antibodies to Ad5 and Ad7 in serum; and 3) Neutralizing antibody assay of serum for inhibition of HIV infection. In addition, replication of the E3-deleted recombinants will be assessed by PCR of stool samples using Adenovirus primers.

These experiments demonstrate that the replication incompetent Ad7 virus elicits an immune response.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

1. An adenovirus serotype 7 cosmid vector comprising an adenovirus serotype 7 genome.

2. The vector of claim 1, wherein the adenovirus serotype 7 genome lacks a functional E1 gene region.

3. The vector of claim 2, wherein all or part of the E1 gene region is deleted.

4. The vector of claim 3, wherein the deleted region includes all or part the coding sequence for protein IX.

5. The vector of claim 2, wherein the vector is further lacking a functional region selected from the group consisting of an E2, E3, and E4 region.

6. The vector of claim 2, wherein all or part of an E3 gene region is deleted.

7. The vector of claim 2, further comprising an expression cassette comprising a nucleic acid sequence encoding a heterologous gene product.

8. The vector of claim 7, wherein the expression cassette comprises a nucleic acid sequence encoding an HIV gene product.

9. The vector of claim 8, wherein the HIV gene product is an envelope polypeptide.

10. The vector of claim 9, wherein the vector comprises an expression cassette that further comprises a nucleic acid sequence encoding a rev gene product.

11. An adenovirus serotype 7 cosmid vector comprising an adenovirus serotype 7 genome which is deleted in all or part of an E1 and an E3 gene region and which further comprises an expression cassette comprising a nucleic acid sequence encoding an HIV gene product.

12. The vector of claim 11, wherein the HIV gene product is an envelope gene.

13. The vector of claim 12, wherein the expression cassette further comprises a nucleic acid sequence encoding an HIV rev gene product.

14. A method of producing a replication defective adenovirus type 7, the method comprising:
transfecting an adenovirus type 7 packaging cell line with a cosmid vector comprising an adenovirus serotype 7 genome lacking a functional E1 gene.

15. The method of claim 14, wherein all of part of the E1 gene is deleted.

16. The method of claim 14, wherein all or part of an E3 gene is deleted.

17. The method of claim 14, further comprising an expression cassette comprising a nucleic acid sequence encoding a heterologous gene product.

18. The method of claim 17, wherein the heterologous gene product is an HIV gene product.

19. The method of claim 18, wherein the HIV gene product is an envelope polypeptide.

20. The method of claim 19, wherein the expression cassette further comprises a nucleic acid sequence encoding an HIV rev gene product.

21. A method of preventing or treating infection with HIV-1, the method comprising administering a replication defective adenovirus comprising an adenovirus type 7 genome lacking a functional E1 gene region, wherein the adenovirus type 7 genome further comprises an expression cassette encoding an HIV-1 gene product.

22. The method of claim 21, wherein the adenovirus type 7 genome is deleted in all or part of the E1 gene region.

23. The method of claim 21, wherein the vector is further lacking a functional region selected from the group consisting of an E2, E3, and E4 region.

24. The method of claim 21, wherein the adnovirus type 7 genome is deleted in all or part of E3.

25. The method of claim 21, wherein the HIV-1 gene product is an env polypeptide.

26. The method of claim 25, wherein the expression cassette further comprises a nucleic acid sequence encoding an HIV rev gene product.

27. The method of claim 21, further comprising administering a replication competent adenovirus.

28. The method of claim 21, further comprising administering an adenovirus of a different serotype.

29. A method of preventing or treating infection with HIV-1, the method comprising administering a replication defective adenovirus, wherein the adenovirus comprises an adenovirus type 7 genome which is deleted in all or part of an E1 gene region and an E3 gene region, and further, wherein the adenovirus type 7 genome comprises an expression cassette comprising a nucleic acid sequence encoding an HIV gene product.

30. A replication defective adenovirus, wherein the adenovirus comprises an adenovirus type 7 genome which is deleted in all or part of an E1 gene region and further, wherein the adenovirus type 7 genome comprises an expression cassette comprising a nucleic acid sequence encoding an HIV gene product.

31. The adenovirus of claim 30, wherein the adenovirus type 7 genome further comprises a deletion of all or part of an E3 gene region.

32. The adenovirus of claim 30, wherein the expression cassette comprises a nucleic acid sequence encoding an HIV env gene product.

33. The adenovirus of claim 32, wherein the expression cassette further comprises a nucleic acid sequenc encoding a rev gene product.
